(19) United States
(12) Patent Application Publication
(10) Pub. No.: US 2005/0147527 A1
(43) Pub. Date: Jul. 7, 2005

(54) Microbial destruction using a gas phase decontaminant

(76) Inventors: I. Brown, Mesa, AZ (US); S. Chenette Carroll, Austin, TX (US); D. Elrod, College Station, TX (US); M. J. Flusche, College Station, TX (US); G. D. Hitchens, College Station, TX (US); R. C. Yalamanchili, College Station, TX (US); W. White, West Lafayette, IN (US)

Correspondence Address: STREETS & STEELE, 13831 NORTHWEST FREEWAY, SUITE 355, HOUSTON, TX, 77040, US

(21) Appl. No.: 10/752,306

(22) Filed: Jan. 6, 2004

(51) Int. Cl. A61L 2/20

(52) U.S. Cl. 422/33; 422/37; 422/34; 422/292

(57) ABSTRACT

An apparatus and method for destroying microorganisms on the surface of an object, the apparatus including a vacuum chamber, and a decontaminant source that provides a decontaminant that may be vaporized and act either as a disinfecting agent, a sanitizing agent or a sterilizing agent. The apparatus further includes a sealable container that contains the object to be decontaminated and that may be used to vacuum pack the object after the decontamination process is complete. The method includes placing an object in a sealable container, drawing a vacuum on the interior and the exterior of the sealable container, injecting a decontaminant into the sealable container and releasing the vacuum.

### BACKGROUND OF THE INVENTION

1. Field of the Invention

The present invention relates to sanitizing systems, and more particularly to apparatus and methods for disinfecting and sterilizing vacuum packed objects.

2. Description of the Related Art

Microbial spoilage is the main determinant of the shelf life of many foodstuffs that are stored at ambient or chill room temperatures. The rate at which microbes act to deteriorate food is governed by many factors. Generally, the number of microbes that are present on the foodstuff when it goes into storage is the single greatest factor that determines the rate of microbial growth, and hence the shelf life of the foodstuff. Therefore, to maximize shelf life, it is important to ensure that the foodstuff is exposed to the lowest concentration of microbes as possible.

Preservation of food products can be achieved using a variety of approaches. Physical manipulations of food products that have a preservative effect include, for example, freezing, refrigerating, cooking, pasteurizing, drying, and vacuum packing and sealing in an oxygen-free package. Some of these approaches can be part of a food processing operation. Food processing steps are typically selected to strike a balance between obtaining a microbial-safe food product and producing a food product with desirable qualities.

Microbial contamination is also a concern in hospitals and in homes. It is critical that, in hospital and dental office environments, medical instruments be sterilized to remove all forms of microbial organisms before the instruments are intrusively used on a patient. In the home, many items used by a baby or used for personal hygiene should be sanitized before use and once sanitized, should be stored in a clean environment until needed.

Medical and dental instruments are often sterilized using high temperature steam. High temperature steam is a very effective sterilant but cannot be used on items that would be damaged by the high temperature, such as foodstuffs and other fragile items. Sterilizers that operate with steam also tend to be prohibitively costly. Other methods of sterilization or disinfection include chemical means which, if used on foodstuffs, would poison the food and make is unsuitable for consumption.

What is needed is a safe efficient method and apparatus that can be used on a wide range of materials, ranging from foodstuffs to medical equipment, to destroy microbial contamination. Such a method and apparatus should be capable of destroying microorganisms at low temperatures without using a chemical that would damage the items being decontaminated. The range of microbial destruction may range from that necessary to provide safe handling of personal hygiene items to medical and dental equipment that must be sterilized for invasive procedures.

### SUMMARY OF THE INVENTION

The present invention provides a method and apparatus for killing microorganisms on an object. The method for killing microorganisms on an objects includes placing an object in a sealable container; drawing a vacuum on an interior and an exterior of the sealable container; placing an object in a sealable container; providing a decontaminant vapor into the sealable container; releasing the vacuum on the exterior of the sealable container; and sealing the sealable container. The step of providing a decontaminant vapor may further include supplying a liquid decontaminant that vaporizes under the vacuum.

The method may further include controlling a pressure differential between the interior and the exterior of the sealable container. In one embodiment, the method includes maintaining a positive pressure differential between the interior and exterior of the sealable container at a positive pressure differential that is great enough to inflate the container, yet low enough to avoid bursting the container. The step of controlling the pressure differential may further comprise venting the interior of the sealable container to the exterior of the sealable container. In some embodiments, the pressure differential may be controlled, for example, between about 0.25 psi and about 3 psi or preferably, between about 0.5 psi and about 1.5 psi.

The decontaminant may be selected from any of the many decontaminants known to those having ordinary skill in the art and may be a sterilant, a disinfectant or a sanitizing decontaminant. The decontaminants may be selected for use singly or in combination with any of the other decontaminants and may even be electrochemically generated on site. One embodiment of the present invention provides hydrogen peroxide as the decontaminant, typically having a concentration as a liquid of between about 5 percent and about 60 percent. Alternatively, the concentration of the liquid hydrogen peroxide may range between about 15 percent to about 50 percent, with a preferred range of between about 30 percent and about 60 percent. Additionally, the decontaminant may be selected from chlorine, chlorine dioxide, ethylene oxide, ozone, or combinations thereof. Alcohols may also be used as the decontaminant as well as organic peroxides, peroxycarboxylic acids or combinations thereof.

When the decontaminant is stored or produced as a liquid, the decontaminant may be provided as a liquid and then vaporized before the step of providing the decontaminant into the sealable container. The liquid may be vaporized by passing the liquid through a flash evaporator or may be vaporized merely through its introduction into an area under the vacuum pressure. Optionally, the liquid may first be heated. The method may further include heating an area surrounding the exterior of the sealable container to reduce condensation of the decontaminant vapor on the walls of the sealable container and optionally may include heating a conduit that delivers the decontaminant to the sealable container to reduce condensation of the decontaminant within the conduit. In some embodiments, the decontaminant is provided into the sealable container at a temperature between about 10° C. and about 95° C. or alternatively, between about 30° C. and about 85° C. or between about 20° C. and about 50° C. These temperatures are not meant to be limiting, however, as the temperature may vary outside these ranges for some applications.

The vacuum pressure drawn on the interior and the exterior of the sealable container may be of any vacuum pressure up to about 29 inches Hg of vacuum. In one embodiment, the range of the vacuum drawn on the interior of the container measures between about 15 and about 29 inches Hg vacuum. In an alternative embodiment, the vacuum drawn on the interior of the container measures between about 10 and about 29 inches Hg vacuum.

Optionally, the method of the present invention may include providing a sterile purge gas into the sealable container. The sterile purge gas may be heated before the gas is used to purge the sealable container.

One embodiment of an apparatus for killing microorganisms on an object includes a vacuum chamber in selective fluid communication with a first vacuum source and a sealable container disposed within the vacuum chamber, wherein the sealable container is in selective fluid communication with a second vacuum source and a decontaminant source. In some embodiments, the first and second vacuum sources may be the same vacuum source, such as, for example, a vacuum pump. The sealable container may further be in selective fluid communication with the vacuum chamber. The sealable container comprises at least one gas port providing the selective communication. The at least one gas port may be, for example, a valve or a septum.

The decontaminant source may be a liquid reservoir or a gas reservoir or both. The decontaminant source may also be an electrochemical generator that produces the decontaminant. The decontaminants that may be used in the apparatus of the present invention are the same as those that may be used in the method of the present invention.

In some embodiments, the apparatus may include a pressure differential indicator sensing pressure differential between the interior of the sealable container and the vacuum chamber. The apparatus may further include a controller that adjusts pressure within the sealable container to control the pressure differential. The controller may be selected from, for example, a digital controller, an analog controller, and combinations thereof.

When the decontaminant is a liquid, the apparatus may further include a flash evaporator, wherein the flash evaporator vaporizes the decontaminant flowing from the decontaminant source to the gas port. The flash evaporator may also operate under a vacuum. A pump may be utilized to pump the decontaminant from the decontaminant source to the flash evaporator.

In some embodiments of the present invention, the sealable container is a flexible bag that may be made, for example, from plastic, nylon or combinations thereof. The sealable container may further be provided in selective fluid communication With the decontaminant source by a conduit inserted through a sealable port in the flexible bag. Optionally, the apparatus of the present invention may include a sterile purge gas source in selective fluid communication with the sealable bag.

The foregoing and other objects, features and advantages of the invention will be apparent from the following more particular description of a preferred embodiment of the invention, as illustrated in the accompanying drawing wherein like reference numbers represent like parts of the invention.

### BRIEF DESCRIPTION OF THE DRAWINGS

FIG. 1 is a schematic diagram of an apparatus that may be used in accordance with one embodiment of the present invention.

FIGS. 2A-2B are schematic views of two different embodiments of the sealable container that may be used in accordance with the present invention.

FIGS. 3A-3B are a flowchart of a method that may be implemented in accordance with the present invention.

### DETAILED DESCRIPTION

The present invention provides methods and apparatus for killing microorganisms that contaminate the surfaces of objects that are contained within a sealable container. After the microorganisms are reduced to a desired level of decontamination, the objects may be vacuum-sealed within the container, thereby sealing the contents against new contamination by microorganisms. In one preferred embodiment, medical or dental equipment or instruments may be sterilized with a gaseous sterilizing agent and then vacuum packed and sealed to prevent further contamination until the medical equipment is needed for its intended use. In another preferred embodiment, foodstuffs may be sanitized with a gaseous sanitizing agent and then vacuum packed and sealed to prevent further contamination until the foodstuffs are needed for consumption. As used herein, the term “gas” includes vapors unless otherwise noted. Many of the embodiments of the present invention utilize an antimicrobial vapor, but the invention is not meant to be limited only to vapors since other gases may also be used.

The destruction of microorganisms that are contaminating the surface of an object occurs at different levels of effectiveness depending upon the required decontamination for a given application. For example, the effectiveness of the destruction of the microorganisms may range between the decontamination levels of sanitizing to disinfecting to sterilizing. Sterilization results in the destruction of virtually all the living forms of the contaminating microorganisms, including spores. Disinfection results in destruction of most of the contaminating microorganisms in their vegetative state but does not destroy all other forms, such as spores. Sanitizing results in the destruction of enough microorganisms to permit safe handling and use as, for example, personal hygiene items, and destroys the least number of the contaminating microorganisms.

The present invention is applicable for many different objects and materials that may be placed within a sealable container, including foodstuffs, surgical instruments, medical equipment, personal hygiene products, kitchen utensils, baby care items and the like. For foodstuffs, including meat and fresh vegetables, the present invention increases the shelf life of the food and minimizes the risk of food poisoning or the passage of microbial infection on to a person consuming the food after it is prepared.

In one embodiment of the invention, one or more items to be decontaminated are placed into a sealable container. The container may have rigid walls or pliable or resilient walls. Vacuum bags having pliable walls that are capable of conforming to the shape of the objects that are vacuum packed within are used as sealable containers in one preferred embodiment. Such bags may be made of any pliable material capable of keeping air or other gases and liquids from leaking back into the bag. Many vacuum bags, for example, are made of a nylon/polyethylene combination material, normally having a thickness of between about 2 mils and about 7 mils. Nylon is preferably used in the bag construction to provide a longer vacuum life because without the nylon, air from the outside atmosphere will refill the bag in just a few hours. Alternatively, a polyethylene bag may be used.

The sealable container also includes a means through which a vacuum may be created to evacuate the sealable container and through which a decontaminant may be injected into the sealable container to destroy the microorganisms. In one embodiment, the means through which a vacuum may be created is a gas port that permits a conduit, such as a tube, pipe, hose or other suitable conduit, to connect to the sealable container. The gas port may be a stopcock, a valve, a septum, or any other sealable opening in the container that permits fluid communication between the conduit and the interior of the sealable container.

Another means through which a vacuum may be created to evacuate the sealable container includes, for example, using a vacuum bag as the sealable container and a conduit having a nozzle that is insertable into the vacuum bag through the bag opening. The bag may then be gathered around the nozzle and mechanically sealed, as with a clamp or wire tie, against the nozzle to prevent leakage from around the nozzle. After the decontamination is complete and the nozzle is removed, the opening may be sealed by melting a strip of the plastic bag. Melting the strip on the bag seals the plastic bag, thereby maintaining the vacuum inside the bag. Alternatively, any means of sealing the bag at the opening is adequate, including mechanical means.

After the objects that are to be decontaminated are placed into the sealable container, the container is sealed and placed into a vacuum chamber and the conduit is connected to the sealed container. A vacuum source is used to cerate a vacuum within the vacuum chamber and within the sealed container. The vacuum source is typically a vacuum pump but any vacuum source is acceptable, including, for example, a vent to outer space if the vacuum chamber is located on a spacecraft. The vacuum pressure created within the vacuum chamber and within the sealed container may be any vacuum pressure up to about 29 inches Hg (mercury) of vacuum. In one embodiment, the vacuum created within the vacuum chamber and within the sealed container may range between about 10 inches Hg of vacuum and about 28 inches Hg of vacuum. In another embodiment, the vacuum may range between about 20 inches Hg of vacuum to about 28 inches Hg of vacuum.

The vacuum is developed within the sealable container through the end of the conduit that is connected, attached, or otherwise in fluid communication with the interior of the sealable container. The second end of the conduit is connected, typically through a valve, directly to the vacuum source or alternatively, to the interior of the vacuum chamber. When the valve is opened, vacuum may be pulled directly from the vacuum source or, in the alternative arrangement, the container may be vented to the vacuum chamber to pull a vacuum within the sealable container.

In one preferred embodiment, the pressure differential between the interior of the sealable container and the vacuum chamber surrounding the sealable container is monitored and controlled to prevent the sealed container from bursting. If the sealable container has flexible walls, as when the sealable container is a bag, it is preferred that the pressure within the sealable container remain higher than the pressure within the vacuum chamber so that the sealable container does not collapse around the object to be decontaminated. With a positive differential pressure, the bag will remain expanded or “puffed up,” so that the side walls of the bag are not touching the object to be decontaminated. Monitoring and control of the pressure differential may be accomplished manually by the user of the apparatus or may be accomplished automatically with either analogue or digital controls, pressure transmitters and control valves as are well known to those having ordinary skill in the art. The pressure differential may be controlled to any value desired, but in one embodiment, the pressure differential may be controlled between about 0.25 psi and about 3 psi or preferably, between about 0.5 psi and about 1.5 psi.

After the vacuum has been drawn within the vacuum chamber and within the sealable container, a decontaminant is injected into the sealable container. The decontaminant may be any suitable decontaminant as known to those having ordinary skill in the art, such as hydrogen peroxide, chlorine, chlorine dioxide, ethylene oxide, ozone, and alcohols, such as n-propanol, benzylalcohol, isopropanol and methanol. Organic peroxides and peroxycarboxylic acids, such as peracetic acid may be used. Organic acids, such as acetic acid, lactic acid, and sorbic acid, are also suitable for use as a decontaminant. Additionally, aldehydes, such as formaldehyde, glutaraldehyde and ortho-phthaldehyde, may be used as the decontaminant as well as phenolic compounds. It should be noted than any of the suitable decontaminants may be used singly or in combinations with each other. Preferably, the decontaminant is injected into the sealable container in a gaseous form through the conduit that is connected or attached to the sealable container. As the decontaminant is injected into the sealable container, the decontaminant flows over the surfaces of the object and destroys the microorganisms that are on the surfaces of the object to be decontaminated. Depending upon the decontaminant used, the concentration of the decontaminant, and the length of time that the decontaminant is allowed to contact the object's surfaces, the degree of decontamination may range from sanitized to disinfected to sterilized.

One preferred decontaminant is hydrogen peroxide. Hydrogen peroxide may be used as a sterilant agent, a disinfectant agent, or a sanitizing agent. One advantage of hydrogen peroxide is that it demonstrates broad-spectrum efficacy against viruses, bacteria, yeasts, and bacterial spores. One advantage of hydrogen peroxide is that it breaks down into water and oxygen, which are safe chemicals that will not damage or contaminate the objects being decontaminated and are safe for human consumption. Hydrogen peroxide may be used as a decontaminant at any concentration, but a concentration in the liquid form of between about 30% and about 60% is preferred, with a concentration of about 50% more preferred.

As the decontaminant is injected into the sealable container, it may occasionally be necessary to vent the sealable container to prevent the sealable container from over-pressuring and bursting. As disclosed above, the pressure differential between the vacuum chamber and the interior of the sealable container may be monitored and controlled to prevent the sealable container from over-pressuring. If the pressure differential is high, injection of the decontaminant may be stopped and a valve on the conduit may be opened to vent the sealable container into the vacuum chamber. Alternatively, a valve may be opened on the conduit that opens to the vacuum source so that the vacuum source can pull additional vacuum on the sealable container. Furthermore, a vent valve on the vacuum chamber may provide automatic or manual means to vent the interior of the vacuum chamber to the exterior of the vacuum chamber. Opening the vent valve would increase the pressure in the vacuum chamber until the interior of the vacuum chamber was equal to the atmospheric pressure outside of the vacuum chamber.

The process of “venting” as described herein should be recognized to include both a partial and a complete venting or relief of pressure. It should also be recognized that the conduit that is connected or attached to the sealable container may comprise multiple sections attached or connected to the sealable container through one or more means. In one embodiment, for example, one section of the conduit may connect the decontaminant source to the sealable container at a first gas port and a second section of the conduit may connect the vacuum source to the sealable container at a second gas port. In this embodiment, the vacuum source may draw the vacuum through the first section of conduit while the decontaminant is being injected through the second section of conduit that is connected to the decontaminant source.

The decontaminant may be stored in a reservoir until being injected into the sealable container. The reservoir may be a liquid or a gaseous reservoir for storing the decontaminant. Preferably, if the decontaninant is stored as a liquid, the decontaninant is pumped or pressured into an evaporator, such as an electrically heated flash evaporator, before being injected into the sealable container as a gas. The decontaminant is preferably heated to a temperature between about 20° C. and about 95° C., most preferably between about 30° C. and about 85° C.

A positive displacement pump is used in one preferred embodiment to ensure injection of a known quantity of decontaminant over a period of time. If the decontaminant is vaporized in an evaporator, preferably the conduit between the evaporator and the sealable container is heated, such as with electric heat tracing, and insulated to prevent the decontaminant from condensing. Optionally, the flash evaporator may also be operated under a vacuum to reduce the temperature required to vaporize the decontaminant.

A rotameter, a positive displacement meter, an orifice plate or capillary tube having a differential pressure indicator, or other flow measurement device that is well known to those having ordinary skill in the art, may measure the decontaminant rate of injection. The decontaminant injection rate may be controlled manually or may be controlled by the digital or analogue controller. If the decontaminant is injected as a liquid, then a nozzle that disperses the liquid as fine droplets when the decontaminant is injected into the sealable container is preferred. The vacuum contained within the sealable container will also help vaporize any liquid decontaminant or maintain a gas decontaminant in the gaseous state. The liquid may be heated before being injected into the sealable container by, for example, using electric heat tracing on the decontaminant conduit or by passing the decontaminant through a heater, such as an electric heater or heat exchanger.

Alternatively, the decontaminant may be produced in an electrochemical cell and used immediately or stored in the reservoir until needed. Especially suitable for electrochemical generation is hydrogen peroxide, ozone, and chlorine dioxide. Methods and apparatus for electrochemical generation of hydrogen peroxide, ozone and chlorine dioxide is well known to those having ordinary skill in the art.

Although multiple objects may be placed in the sealable container, it is preferred that numerous objects are not placed in the same sealable container for decontamination unless provisions are made to ensure that the decontaminate will reach all the surfaces of the objects. Since decontamination can only occur if the decontaminant contacts the contaminated surfaces, if multiple objects are placed in the sealable container and touch each other, the touching surfaces will block the decontaminant from effectively reaching those object surfaces. Likewise, it is preferred that the object to be decontaminated be placed on supports when necessary to prevent the object from lying upon the inside wall of the sealable container, thereby blocking the decontaminant from contacting the object surfaces that are contacting the inside wall. As one provision to ensure adequate contact of the surfaces with the decontaminate, the object or objects may be turned or otherwise moved during the decontamination process to allow contact of all surfaces with the decontaminant.

After the decontamination process is completed and the object within the sealable container has been sanitized, disinfected or sterilized, the sealable container may be vented to the vacuum chamber or to the vacuum source to provide vacuum packaging, and then the vacuum chamber may be returned to atmospheric pressure. The sealable container is removed from the vacuum chamber, the gas port closed and sealed and the conduit removed. The sealable container may then be stored in a sealed condition until the contents of the sealable container are needed. The sealable container provides storage of the contents in a protected environment against new contamination by microorganisms.

In one embodiment, the method of the present invention may include purging the sealable container with a sterile purge gas following the decontamination process. A sterile purge gas may be provided and optionally heated before purging the container. The sterile purge gas may be heated by passing the purge gas through a conduit having electric heat tracing, or by passing the purge gas through an electric heater or heat exchanger. Purging the sealable container with a sterile gas removes any decontaminate vapor or other vapors remaining in the container that may later condense and/or removes condensate or other liquids that may have already formed within the sealable container.

Heated sterile air that has been filtered of any microbiological contaminants may, for example, could be used as the sterile purge gas. Sterile nitrogen is another example of a suitable sterile purge gas. The sterile purge gas may, for example, be stored in a gas reservoir and provided to the sealable container through the same conduit that provides the decontaminant or, alternatively, may be provided through a separate conduit. Solenoid valves, other control valves, and flow measurement devices may be used to control the sterile gas flow or the flow may be adjusted manually. The pressure differential between the interior and exterior of the sealable container may be controlled in the same manner during the sterile gas purge as it is controlled during the decontaminant step as described previously. Optionally, if the object is not to be vacuum packed for storage in the sealable container, the sterile gas purge may be used to break the vacuum and the sealable container may be stored under a slight positive pressure of the sterile gas.

One advantage of the present invention over most vapor phase hydrogen peroxide systems is that the hydrogen peroxide is used under vacuum, which minimizes the risk of explosion that is inherent in the use of hydrogen peroxide. Embodiments of the present invention may also include level alarms and/or system shutdowns for high and low levels of decontaminant in the decontaminant reservoir to protect against operator error. When using flammable or explosive decontaminants with a flash evaporator, high temperature shutdowns of the flash evaporator may provide an additional safety feature.

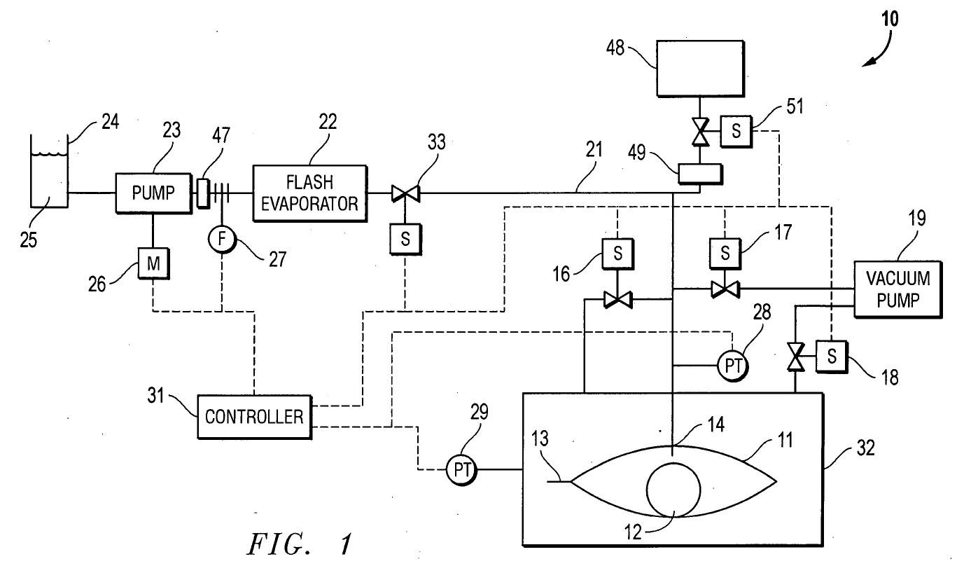

FIG. 1 is a schematic diagram of an apparatus that may be used in accordance with one embodiment of the present invention. The decontamination apparatus 10 includes a vacuum chamber 32 connected to a vacuum pump 19. A solenoid valve 18 can be opened and closed by the controller 31 to allow the vacuum pump 19 to pull more or less vacuum on the vacuum chamber 32. A sealable container 11 is placed within the vacuum chamber 32 and a conduit 21 is attached to a closeable or sealable gas port 14, such as a valve, on the sealable container 11. The object 12 to be decontaminated is sealed within the sealable container 11. The seal 13 may be formed by mechanical means or preferably, by melting or plastic welding the plastic sides of the sealable container 11 together.

A pressure transmitter 28 measures the vacuum within the sealable container 11 and a second pressure transmitter 29 measures the vacuum within the vacuum chamber 32. A controller 31 monitors the two pressure transmitters 29, 28 and determines the differential pressure between the sealable container 11 and the vacuum chamber 32. Alternatively, a single differential pressure transmitter may replace the two pressure transmitters 29, 28. If the pressure differential becomes too great so that the sealable container is in danger of bursting, the controller may open a vent solenoid valve 16 to vent the sealable container 11 into the vacuum chamber 32, thereby tending to equalize the pressures and reduce the pressure differential. Alternatively, the controller 31 may open a suction solenoid valve 17 to the vacuum pump to vent the sealable container 11 through the vacuum pump 19. It is desirable to keep the pressure within the sealable container 11 higher than the pressure within the vacuum chamber 11 so that the sealable bag 11 stays “inflated” and the sides of the internal walls of the sealable container 11 do not touch the object to be decontaminated 12.

Decontaminant 25 is pumped from a decontaminant reservoir 24 to the sealable container 11. A pump 23 pumps the decontaminant 25 to an electrically heated flash evaporator 22 to vaporize the decontaminant 25 before it is injected into the sealable container 11. The amount of decontaminant 25 pumped to the flash evaporator may be controlled by a variable speed motor 26 that is adjusted by the controller 31. A flow measurement device 27 may be monitored by the controller 31 to monitor and control the amount of disinfectant flowing through the flow indicator 27 to the flash evaporator 22.

The hydrogen peroxide delivery system is preferably self-priming and designed to optionally remove “old” hydrogen peroxide left in the conduits from previous cycles of use. The conduits made of various materials, but are preferably stainless steel since polymeric tubing may degrade over time due to exposure to hydrogen peroxide.

ZIPLOC (a trademark of S.C. Johnson & Son, Inc.) polyethylene bags can be used. One-gallon bags were used. The ZIPLOC freezer bags are modified to include a commercially available TEFLON (a trademark of DuPont) PTFE miniature stopcock valve that can be extended through a hole cut in the bag and mechanically sealed against the bag, preferably with an o-ring and the bag wall being compressed between two plates coupled by a threaded connection. The bags are preferably used only once and discarded after storage and use of the disinfected object. However, the stopcock valves may be reused many times through cleaning and reattachment to a new bag.

The controller 31 is most preferably a personal computer running LABVIEW (a trademark of National Instruments) software. However, it should be recognized that the controller may be based on analog circuitry, digital circuitry, or some combination thereof and that the controller may be a single device or multiple devices with central or distributed control.

The conduit between the displacement pump and the vacuum chamber will preferably include one or more filters to remove particulates from the liquid hydrogen peroxide that could detrimentally affect the accurate delivery or measurement of the hydrogen peroxide. In particular, a filter 47 is desirably positioned in the conduit before passing the hydrogen peroxide solution through an orifice plate or other restriction used to cause a sufficient pressure drop to foster vaporization of the hydrogen peroxide alone or in combination with heat.

Optionally, the system may include a sterile purge gas source 48. The sterile purge gas source 48 may be, for example, a gas cylinder. A solenoid valve 51 is opened by the controller 31 to purge the decontaminant 25 from the sealable container 11 after the object 12 has been decontaminated. The controller 31 may control the pressure differential between the vacuum chamber 32 and the interior of the sealable container 11 as described herein during the step of injecting the decontaminant 25.

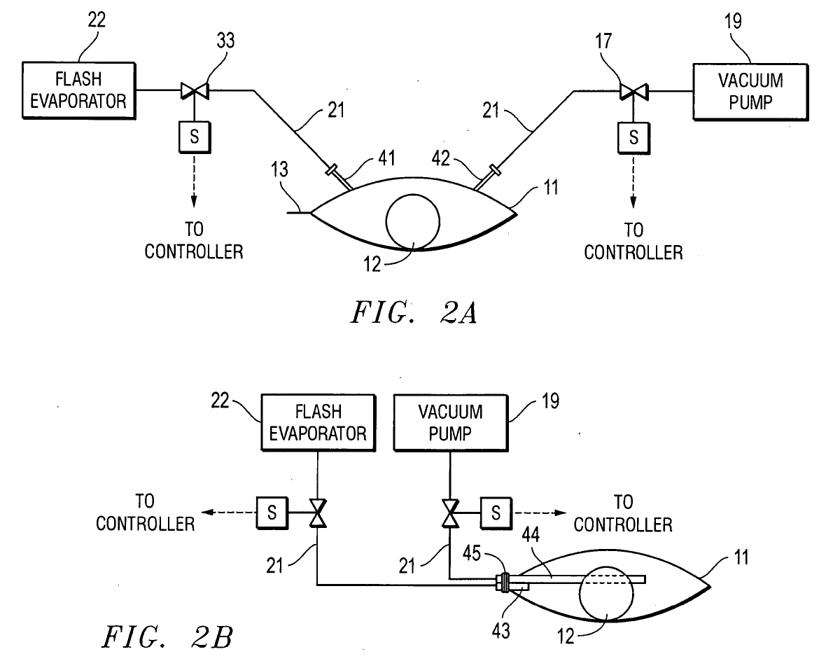

FIGS. 2A-2B are schematic views of two different embodiments of the sealable container that may be used in accordance with the present invention. In FIG. 2A, a sealable container 11 is shown having two gas ports, 41, 42. One gas port 41 provides fluid communication between the interior of the sealable container 11 and the flash evaporator 22. The second gas port 42 provides fluid communication between the sealable container 11 and the vacuum pump 19. This embodiment allows the vacuum pump 19 to evacuate the sealable container 11 from the gas port 42 that is opposite to the end of the sealable container into which the decontaminant is injected through the second gas port 41. Accordingly, the decontaminant may be more controllably drawn into and across the interior of the bag.

In FIG. 2B, an embodiment of the sealable container 11 is shown having two nozzles 43, 44 inserted into the opening of the sealable container 11 with a clamp 45, sealing the opening of the sealable container 11 against the two nozzles 43, 44. One of the nozzles, a long nozzle 44, extends to the opposing end of the sealable container 11, whereas the other nozzle, a short nozzle 43, extends just past the opening of the sealable container 11. The long nozzle 44 is attached to a conduit 21 that provides suction to the vacuum pump 19 and the short nozzle 43 is attached to the conduit 21 that provides disinfectant from the flash evaporator 22. This embodiment provides the same advantage as the sealable container embodiment shown in FIG. 2A so that the long nozzle pulls a vacuum from the opposite end of the sealable container into which the decontaminant is injected. It should be recognized that the same advantages are obtained connecting the long nozzle to the flash evaporator and the short nozzle to the vacuum pump.

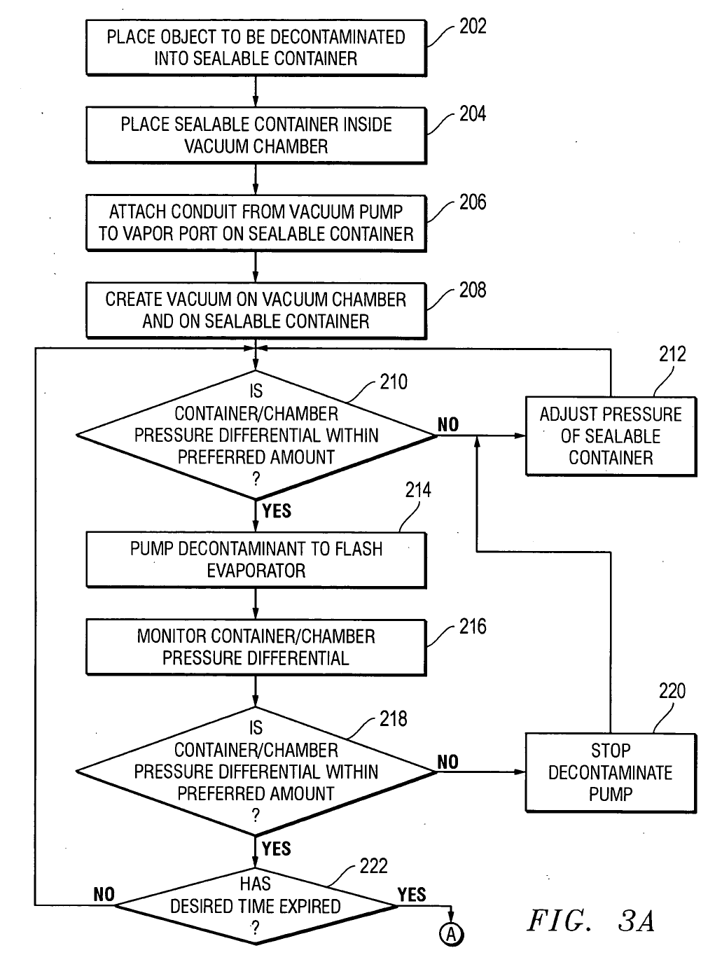

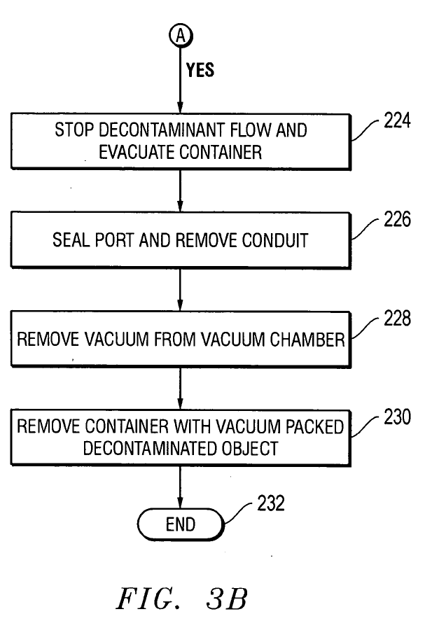

FIGS. 3A-3B are a flowchart of a method that may be implemented in accordance with the present invention. In state 202, the object to be decontaminated is placed into a sealable container. In state 204, the sealable container is placed inside a vacuum chamber. In state 206, a conduit from the vacuum pump is attached to the gas port of the sealable container. In state 208, the vacuum pump is started and a vacuum is created within the vacuum chamber and within sealable container. In state 210, the controller determines whether the differential pressure between the sealable container and the vacuum chamber is at the desired amount. If, in state 210, the differential pressure is not within the desired value, then in state 212, the pressure of the sealable container or the vacuum chamber is adjusted and the method returns to state 210. If, in state 210, the differential pressure is at the desired amount, then in state 214, the decontaminant pump is started and decontaminant is pumped into the flash evaporator.

In state 216, the system continues to monitor the differential pressure between the sealable container and the vacuum chamber. If, in state 218, the differential pressure is not within the desired range, then in state 220, the decontaminate pump is shut off and the method continues to state 212 as discussed above. If, in state 218, the differential pressure is within the desired range, then the method continues to step 222. It should be noted that if separate conduits are used for fluid communication between the vacuum pump and the sealable container and between the flash evaporator and sealable container (as shown in FIGS. 2A and 2B), then the pressure differential between the vacuum chamber and the interior of the sealable container may be adjusted without having to first turn the decontaminate pump off, or otherwise stop the flow of decontaminant to the sealable container.

If, in state 222, the desired decontamination time has not expired, then the method continues to state 210 as discussed above. If, in state 222, the desired decontamination time has expired, then in state 224, the decontaminant flow is shut off and the sealable container is fully evacuated. In state 226, the port to the sealable container is closed or sealed and the conduit removed from the sealable container. In state 228, the vacuum is released from the vacuum chamber and in state 230, the container is removed from the vacuum chamber with the object sealed and vacuum packed within the container. In state 232, the method ends. Alternatively, states 226 and 228 may be reversed with the advantage being that a higher pressure in the vacuum chamber may assist in expelling gas from the sealable container before it is sealed.

### EXAMPLE 1

### Disinfection Efficacy

The following experiments were performed using a system in accordance with the present invention. Food crops were pruned of inedible roots, leaves, etc., and weighed. A given food crop was then inoculated with a single challenge microorganism. All challenge microorganisms were purchased from ATCC as streptomycin-resistant strains-Esherichia coli (E. coli), Staphylococcus aureus (S. aureus), and Salmonella choleraesuis (S. choleraesuis). This was done so that when plating was performed on agar containing streptomycin, any natural flora that contaminated the food upon purchase would be eradicated by the antibiotic thereby allowing only the specific challenge microbe used the opportunity to grow on the plates. After inoculation, the crops were left to dry for 30 minutes to 1 hour. The crops were then placed in the Ziploc® bag and into the vacuum chamber. The bag was then connected to the metal hydrogen peroxide dispensing line via a tubing connector, the desiccator lid was securely put in place, and the disinfection cycle started using the desktop computer and accompanying software. Parameters such as vacuum pressure, dispensing time, and sterilization wait time were varied in order to arrive at some conclusion concerning the optimum conditions for effective disinfection of the food crops. After the disinfection cycle had ended, the crops were neutralized in 50 mL of 10% thiosulfate solution contained in a sterile mastication bag.

The bag was then placed in a masticator and pulverized for 2 minutes. The liquid contents were then serially diluted and plated onto the appropriate agar medium. The plates were incubated at 37° C. for 24 hours and plate counts were performed. A control experiment was performed for each set of conditions, food crop, and microbial challenge using a similar protocol, without running the disinfection cycle. A log reduction in terms of CFU/g was calculated by subtracting the plate counts of the disinfected food from that of its respective control, as shown in Table 1.

TABLE 1
Vapor Phase Disinfection of Various Food Crops

| | Food | | | Log | Log Post- | Log |
|---|---|---|---|---|---|---|
| Food | Weight | Challenge | System | Control | Disinfection | Reduction |
| Crop | (g) | Microorganism | Parameters* | (CFU/g) | (CFU/g) | (CFU/g) |
| Carrots | 80.03 | E. coli | VP: 15 | 4.90 | 0 | 4.90 |
| | | | DT: 18 | | | |
| | | | SW: 30 | | | |
| Carrots | 55.40 | E. coli | VP: 18 | 5.12 | 2.05 | 3.07 |
| | | | DT: 18 | | | |
| | | | SW: 30 | | | |
| Carrots | 62.62 | E. coli | VP: 18 | 5.12 | 1.99 | 3.13 |
| | | | DT: 18 | | | |
| | | | SW: 30 | | | |
| Carrots | 56.35 | E. coli | VP: 18 | 5.12 | 0 | 5.12 |
| | | | DT: 18 | | | |
| | | | SW: 30 | | | |
| Carrots | 81.56 | E. coli | VP: 15 | 5.45 | 0 | 5.45 |
| | | | DT: 18 | | | |
| | | | SW: 20 | | | |
| Carrots | 83.56 | E. coli | VP: 15 | 5.45 | 0 | 5.45 |
| | | | DT: 18 | | | |
| | | | SW: 20 | | | |
| Carrots | 80.48 | E. coli | VP: 15 | 4.90 | 0 | 4.90 |
| | | | DT: 18 | | | |
| | | | SW: 15 | | | |
| Carrots | 78.38 | E. coli | VP: 15 | 4.90 | 0 | 4.90 |
| | | | DT: 18 | | | |
| | | | SW: 10 | | | |
| Carrots | 83.75 | E. coli | VP: 18 | 5.45 | 0 | 5.45 |
| | | | DT: 18 | | | |
| | | | SW: 10 | | | |
| Carrots | 74.25 | E. coli | VP: 17 | 5.45 | 0 | 5.45 |
| | | | DT: 18 | | | |
| | | | SW: 5 | | | |
| Lettuce | 20.03 | E. coli | VP: 15 | 6.38 | 0 | 6.38 |
| | | | DT: 18 | | | |
| | | | SW: 30 | | | |
| Lettuce | 28.41 | E. coli | VP: 15 | 6.24 | 4.10 | 2.14 |
| | | | DT: 18 | | | |
| | | | SW: 30 | | | |
| Lettuce | 24.29 | E. coli | VP: 15 | 6.24 | 1.93 | 4.31 |
| | | | DT: 18 | | | |
| | | | SW: 15 | | | |
| Lettuce | 23.36 | E. coli | VP: 15 | 6.38 | 0 | 6.38 |
| | | | DT: 18 | | | |
| | | | SW: 10 | | | |
| Lettuce | 20.78 | S. aureus | VP: 15 | 6.81 | 2.41 | 4.40 |
| | | | DT: 18 | | | |
| | | | SW: 30 | | | |
| Lettuce | 18.27 | S. aureus | VP: 15 | 6.81 | 1.99 | 4.82 |
| | | | DT: 18 | | | |
| | | | SW: 30 | | | |
| Lettuce | 16.53 | S. aureus | VP: 15 | 6.81 | 1.39 | 5.42 |
| | | | DT: 18 | | | |
| | | | SW: 30 | | | |
| Lettuce | 23.98 | S. aureus | VP: 15 | 6.81 | 0 | 6.81 |
| | | | DT: 18 | | | |
| | | | SW: 15 | | | |
| Radishes | 69.75 | E. coli | VP: 15 | 5.76 | 0 | 5.76 |
| | | | DT: 18 | | | |
| | | | SW: 30 | | | |
| Radishes | 61.03 | E. coli | VP: 15 | 5.76 | 0.42 | 5.34 |
| | | | DT: 18 | | | |
| | | | SW: 30 | | | |
| Radishes | 67.62 | E. coli | VP: 15 | 5.80 | 2.67 | 3.13 |
| | | | DT: 18 | | | |
| | | | SW: 30 | | | |
| Radishes | 70.18 | E. coli | VP: 15 | 5.80 | 3.66 | 2.14 |
| | | | DT: 18 | | | |
| | | | SW: 30 | | | |
| Radishes | 63.00 | E. coli | VP: 15 | 5.35 | 2.01 | 3.04 |
| | | | DT: 18 | | | |
| | | | SW: 30 | | | |
| Radishes | 72.64 | E. coli | VP: 15 | 5.35 | 1.26 | 3.54 |
| | | | DT: 18 | | | |
| | | | SW: 30 | | | |
| Radishes | 68.32 | E. coli | VP: 15 | 5.35 | 1.59 | 3.76 |
| | | | DT: 18 | | | |
| | | | SW: 30 | | | |
| Radishes | 72.65 | E. coli | VP: 18 | 5.23 | 0 | 5.23 |
| | | | DT: 18 | | | |
| | | | SW: 20 | | | |
| Radishes | 70.29 | E. coli | VP: 18 | 5.23 | 1.04 | 4.19 |
| | | | DT: 18 | | | |
| | | | SW: 20 | | | |
| Radishes | 74.15 | E. coli | VP: 18 | 5.23 | 0 | 5.23 |
| | | | DT: 18 | | | |
| | | | SW: 20 | | | |
| Radishes | 67.75 | E. coli | VP: 15 | 5.80 | 3.73 | 2.07 |
| | | | DT: 18 | | | |
| | | | SW: 15 | | | |
| Radishes | 67.33 | E. coli | VP: 15 | 5.80 | 2.85 | 2.95 |
| | | | DT: 18 | | | |
| | | | SW: 10 | | | |
| Radishes | 57.14 | S. aureus | VP: 15 | 6.33 | 0 | 6.33 |
| | | | DT: 18 | | | |
| | | | SW: 30 | | | |
| Radishes | 68.64 | S. aureus | VP: 20 | 6.09 | 3.17 | 2.92 |
| | | | DT: 18 | | | |
| | | | SW: 30 | | | |
| Radishes | 43.22 | S. aureus | VP: 20 | 6.09 | 1.23 | 4.86 |
| | | | DT: 18 | | | |
| | | | SW: 30 | | | |
| Radishes | 64.19 | S. aureus | VP: 20 | 6.09 | 1.61 | 4.48 |
| | | | DT: 18 | | | |
| | | | SW: 15 | | | |
| Radishes | 57.20 | S. aureus | VP: 20 | 6.09 | 0 | 6.09 |
| | | | DT: 18 | | | |
| | | | SW: 10 | | | |
| Radishes | 43.50 | S. aureus | VP: 20 | 6.09 | 0 | 6.09 |
| | | | DT: 18 | | | |
| | | | SW: 5 | | | |
| Radishes | 52.02 | S. aureus | VP: 20 | 6.09 | 0 | 6.09 |
| | | | DT: 18 | | | |
| | | | SW: 5 | | | |

*VP = vacuum pressure (inch Hg);
DT = dispensing time (min), (delivers ˜0.67 mL/min of hydrogen peroxide);
SW = sterilization wait time (min);
Block heater and heated dispensing line were maintained at 180° F. for all runs.

As shown in Table 1, no matter the food crop, the microbial challenge, or disinfection parameters, the log reduction was never below 2 and rarely was a 2-log reduction even observed. In fact, the average log reduction of all of the runs shown in Table 1 was 4.63. This verifies the efficacy of the system as a potent vapor phase disinfection device.

Table 2 shows the results of an experiment to optimize the dispense time of the decontaminant. Lettuce was used as the food crop while streptomycin-resistant E. coli was chosen as the challenge microorganism. The minimum dispense time that gave consistent results was 3 minutes. This dispense time was then used as the optimum for 25-65 grams of a given food crop. Each food crop was inoculated with streptomycin-resistant strains of E. coli, S. aureus, and S. choleraesuis in separate experiments and 3 minutes was used as the dispense time in every run. As shown in Table 3, greater than 3-log reductions for every combination of food crop and microbial challenge was achieved.

TABLE 2
Optimization of the Disinfecting Efficacy against E. coli on Lettuce

| | | Log₁₀ Post- | Log₁₀ |
|---|---|---|---|
| Dispense | Log₁₀ Control | Disinfection | Reduction |
| Time (min) | (CFU/g) | (CFU/g) | (CFU/g) |
| a: 3 | 6.14 | 0 | 6.14 |
| b: 3 | 6.14 | 0 | 6.14 |
| c: 3 | 6.14 | 0 | 6.14 |
| d: 3 | 6.14 | 0 | 6.14 |
| e: 3 | 6.14 | 3.13 | 3.01 |
| f: 3 | 6.14 | 0 | 6.14 |
| a: 2 | 6.26 | 0 | 6.26 |
| b: 2 | 6.26 | 1.71 | 4.55 |
| c: 2 | 6.26 | 3.61 | 2.65 |
| d: 2 | 6.26 | 0 | 6.26 |
| e: 2 | 6.26 | 5.79 | 0.47 |
| f: 2 | 6.26 | 2.16 | 4.1 |

*Boiler Temp = 180° F.,
Vacuum Pressure 18 inch Hg,
Sterilization Wait Time = 0 minutes

TABLE 3
Disinfection Results

| | | | Dispense | Log₁₀ | Log₁₀ Post- | Log₁₀ |
|---|---|---|---|---|---|---|
| Food | | Challenge | Time | Control | Disinfection | Reduction |
| Crop | Weight (g) | Organism | (min) | (CFU/g) | (CFU/g) | (CFU/g) |
| Lettuce | 24.43 | E. coli | 3 | 6.23 | 0 | ≧6.23 |
| Lettuce | 27.41 | E. coli | 3 | 6.23 | 0 | ≧6.23 |
| Lettuce | 27.84 | E. coli | 3 | 6.23 | 0 | ≧6.23 |
| Carrots | 64.47 | E. coli | 3 | 5.90 | 0 | ≧5.90 |
| Carrots | 59.86 | E. coli | 3 | 5.90 | 0.26 | 5.64 |
| Carrots | 54.05 | E. coli | 3 | 5.90 | 0.30 | 5.60 |
| Radishes | 59.00 | E. coli | 3 | 5.25 | 1.83 | 3.42 |
| Radishes | 52.89 | E. coli | 3 | 5.25 | 2.03 | 3.22 |
| Radishes | 64.81 | E. coli | 3 | 5.25 | 2.15 | 3.10 |
| Carrots | 51.86 | S. choleraesuis | 3 | 6.37 | 0.90 | 5.47 |
| Carrots | 44.20 | S. choleraesuis | 3 | 6.37 | 0 | ≧6.37 |
| Carrots | 53.62 | S. choleraesuis | 3 | 6.37 | 0.78 | 5.59 |
| Radishes | 35.91 | S. choleraesuis | 3 | 6.85 | 1.67 | 5.18 |
| Radishes | 35.61 | S. choleraesuis | 3 | 6.85 | 1.95 | 4.90 |
| Radishes | 51.73 | S. choleraesuis | 3 | 6.85 | 1.82 | 4.76 |
| Carrots | 61.18 | S. aureus | 3 | 5.59 | 0.53 | 5.06 |
| Carrots | 60.36 | S. aureus | 3 | 5.59 | 1.60 | 3.99 |
| Carrots | 57.57 | S. aureus | 3 | 5.59 | 1.49 | 4.10 |
| Radishes | 39.00 | S. aureus | 3 | 5.25 | 0 | ≧5.25 |
| Radishes | 60.59 | S. aureus | 3 | 5.25 | 0 | ≧5.25 |
| Radishes | 57.43 | S. aureus | 3 | 5.25 | 0 | ≧5.25 |

*Boiler Temp = 180° F.,
Vacuum Pressure 18 inch Hg,
Sterilization Wait Time = 0 minutes

A protocol analogous to that used for the food crops was employed for spore testing on stainless steel coupons. 3-1.75 cm×3.75 cm stainless steel coupons were each inoculated (front & back) with a total of 100 μL of Bacillus subtilis spores. The spores were allowed to dry and the three coupons were aseptically transferred to a Ziploc® bag. The remaining protocol used was similar to that of the food crops except that each coupon was neutralized in 8 mL of 10% thiosulfate solution and each coupon's solution was serially diluted and plated onto media. Table 4 shows three runs of the spore experiment (Run 1=a₁, a₂, a₃; Run 2=b₁, b₂, b₃; Run 3=c₁, c₂, c₃). As shown, the vapor phase system was able to completely eradicate spores within a 12 minute dispense time. This further shows the potency of the system as spores are very hardy organisms.

TABLE 4
Efficacy of the Vapor Phase Hydrogen
Peroxide System against Spore*

| | | Log₁₀ Post- | Log₁₀ |
|---|---|---|---|
| Dispense | Log₁₀ Control | Disinfection | Reduction |
| Time (min) | (CFU/cm²) | (CFU/cm²) | (CFU/cm²) |
| a₁ | 5.42 | 0 | 5.42 |
| a₂ | 5.42 | 0 | 5.42 |
| a₃ | 5.42 | 0 | 5.42 |
| b₁ | 5.42 | 0 | 5.42 |
| b₂ | 5.42 | 0 | 5.42 |
| b₃ | 5.42 | 0 | 5.42 |
| c₁ | 5.42 | 0 | 5.42 |
| c₂ | 5.42 | 0 | 5.42 |
| c₃ | 5.42 | 0 | 5.42 |

*Boiler Temp = 180° F.,
Vacuum Pressure 18 inch Hg,
Sterilization Wait Time = 0 minutes

Because food crops treated by the system of the present invention will eventually be handled and consumed, it was important to establish how much time was needed to allow any residual hydrogen peroxide to breakdown by further reaction with the food crops or by natural chemical decomposition. Therefore, an experiment (utilizing the optimum parameters previously shown) was performed in a worst case scenario where the food crop (lettuce) was not inoculated and therefore more hydrogen peroxide would persist due to the lack of microorganisms that would consume it. The lettuce would only contain natural microflora present on crops at purchase. The values in Table 5 represent all of the hydrogen peroxide residual remaining in the bag, including that which had settled on the walls and other areas of the bag and not necessarily on the lettuce leaves. The concentration of hydrogen peroxide was determined using a standard potassium permanganate titration after removing the residual hydrogen peroxide from the leaves with deionized water.

TABLE 5
Persistence of Hydrogen Peroxide on Lettuce and Bag over Time

| Storage Time after | Concentration of Hydrogen Peroxide |
|---|---|
| Disinfection (hr) | Remaining on Lettuce and in Bag (%) |
| 24 | 6 |
| 48 | 3-4 |
| 72 | 1-2 |

The terms “comprising,” “including,” and “having,” as used herein, shall be considered as indicating an open group that may include other elements not specified. The terms “a,” “an,” and the singular forms of words shall be taken to include the plural form of the same words, such that the terms mean that one or more of something is provided. The terms “preferably,” “preferred,” and “may” are used to indicate that the item, condition or step being referred to is an optional (not required) feature or limitation of the invention.

It will be understood from the foregoing description that various modifications and changes may be made in the preferred embodiment of the present invention without departing from its true spirit. It is intended that this description is for purposes of illustration only and should not be construed in a limiting sense. The scope of this invention should be limited only by the language of the following claims.

1. A method for killing microorganisms on an object, comprising:
placing an object in a sealable container;
drawing a vacuum on an interior and an exterior of the sealable container;
providing a decontaminant gas into the sealable container;
releasing the vacuum on the exterior of the sealable container; and
sealing the sealable container.

2. The method of claim 1, wherein the step of providing a decontaminant gas includes supplying a liquid decontaminant that vaporizes under the vacuum.

3. The method of claim 1, further comprising;
controlling a pressure differential between the interior and the exterior of the sealable container.

4. The method of claim 1, further comprising:
maintaining a positive pressure differential between the interior and exterior of the sealable container.

5. The method of claim 4, wherein the positive pressure differential is great enough to inflate the container, yet low enough to avoid bursting the container.

6. The method of claim 4, wherein the positive pressure differential is between about 0.25 psi and about 3 psi.

7. The method of claim 4, wherein the positive pressure differential is between about 0.25 psi and about 3 psi

8. The method of claim 3, wherein the step of controlling the pressure differential further comprises:
venting the interior of the sealable container to the exterior of the sealable container.

9. The method of claim 1, wherein the decontaminant is hydrogen peroxide.

10. The method of claim 1, wherein the decontaminant is selected from chlorine, chlorine dioxide, ethylene oxide, ozone, or combinations thereof.

11. The method of claim 1, wherein the decontaminant is an alcohol.

12. The method of claim 1, wherein the decontaminant is selected from an organic peroxide, peroxycarboxylic acids or combinations thereof.

13. The method of claim 1, wherein the decontaminant is selected from organic acids, aldehydes, phenols or combinations thereof.

14. The method of claim 1, wherein the decontaminant is stored as a liquid.

15. The method of claim 14, further comprising:
vaporizing the decontaminant before the step of providing the decontaminant into the sealable container.

16. The method of claim 1, wherein the decontaminant is a sterilant.

17. The method of claim 1, wherein the decontaminant is a disinfectant.

18. The method of claim 1, wherein the vacuum drawn on the interior of the container measures between about 15 and about 29 inches Hg vacuum.

19. The method of claim 1, wherein the vacuum drawn on the interior of the container measures between about 10 and about 29 inches Hg vacuum.

20. The method of claim 1, wherein the vacuum drawn on the interior of the container measures up to about 29 inches Hg of vacuum.

21. The method of claim 1, wherein the decontaminant is hydrogen peroxide having a concentration of between about 5 percent and about 60 percent.

22. The method of claim 1, wherein the decontaminant is hydrogen peroxide having a concentration of between about 15 percent and about 50 percent.

23. The method of claim 1, wherein the decontaminant is hydrogen peroxide having a concentration of between about 30 percent and about 60 percent.

24. The method of claim 1, further comprising;
electrochemically generating the decontaminant.

25. The method of claim 1, wherein the decontaminant is selected from chlorine, ethylene oxide, alcohols, percarboxylic acids, and combinations thereof

26. The method of claim 1, further comprising:
heating an area surrounding the exterior of the sealable container to reduce condensation of the decontaminant vapor on walls of the sealable container.

27. The method of claim 1, further comprising:
heating a conduit that delivers the decontaminant to the sealable container to reduce condensation of the decontaminant within the conduit.

28. The method of claim 1, wherein the decontaminant is provided into the sealable container at a temperature between about 10° C. and about 95° C.

29. The method of claim 1, wherein the decontaminant is provided into the sealable container at a temperature between about 30° C. and about 85° C.

30. The method of claim 1, further comprising:
providing a sterile purge gas into the sealable container.

31. The method of claim 30, further comprising:
heating the sterile purge gas.

32. An apparatus for killing microorganisms on an object, comprising:
a vacuum chamber in selective fluid communication with a first vacuum source;
a sealable container disposed within the vacuum chamber, wherein the sealable container is in selective fluid communication with a second vacuum source and a decontaminant source.

33. The apparatus of claim 32, wherein the sealable container is in selective fluid communication the vacuum chamber.

34. The apparatus of claim 32, wherein the first and second vacuum sources are the same vacuum source.

35. The apparatus of claim 34, wherein the vacuum source is a vacuum pump.

36. The apparatus of claim 32, wherein the decontaminant source is a liquid reservoir.

37. The apparatus of claim 32, wherein the decontaminant source is a gas reservoir.

38. The apparatus of claim 32, wherein the decontaminant source is an electrochemical generator.

39. The apparatus of claim 38, wherein the decontaminant is selected from hydrogen peroxide, ozone, and combinations thereof.

40. The apparatus of claim 32, wherein the decontaminant is selected from hydrogen peroxide, ozone, and combinations thereof.

41. The apparatus of claim 32, wherein the sealable container comprises at least one gas port providing the selective communication.

42. The method of claim 41, wherein the at least one gas port is a valve.

43. The apparatus of claim 41, wherein the at least one gas port is a septum.

44. The apparatus of claim 32, further comprising:
a pressure differential indicator sensing pressure differential between the interior of the sealable container and the vacuum chamber.

45. The apparatus of claim 44, further comprising:
a controller that adjusts pressure within the sealable container to control the pressure differential.

46. The apparatus of claim 45, wherein the controller is selected from a digital controller, an analog controller, and combinations thereof.

47. The apparatus of claim 32, wherein the decontaminant is a liquid, the apparatus further comprises:
a flash evaporator, wherein the flash evaporator vaporizes the decontaminant flowing from the decontaminant source to the gas port.

48. The apparatus of claim 47, wherein the flash evaporator operates under a vacuum.

49. The apparatus of claim 47, wherein the decontaminant is a liquid, the apparatus further comprises:
a pump, wherein the pump pumps the decontaminant from the decontaminant source to the flash evaporator.

50. The apparatus of claim 32, wherein the sealable container is a flexible bag.

51. The apparatus of claim 50, wherein the flexible bag is made of a material selected from plastic, nylon or combinations thereof.

52. The apparatus of claim 50, wherein the sealable container is provided in selective fluid communication with the decontaminant source by a conduit inserted through a sealable port in the flexible bag.

53. The apparatus of claim 32, further comprising a sterile purge gas source in selective fluid communication with the sealable container.
